(19) United States
(12) Patent Application Publication
(10) Pub. No.: US 2020/0286585 A1
(43) Pub. Date: Sep. 10, 2020

(54) RNA-SEQ QUANTIFICATION METHOD FOR ANALYSIS OF TRANSCRIPTIONAL ABERRATIONS

(71) Applicant: KING ABDULLAH UNIVERSITY OF SCIENCE AND TECHNOLOGY, Thuwal (SA)

(72) Inventors: Xin GAO, Thuwal (SA); Hiroyuki KUWAHARA, Thuwal (SA)

(21) Appl. No.: 16/803,122

(22) Filed: Feb. 27, 2020

Related U.S. Application Data
(60) Provisional application No. 62/813,301, filed on Mar. 4, 2019.

(51) Int. Cl. G16B 20/20; G06F 17/18; G16B 50/30

(52) U.S. Cl. CPC G16B 20/20; G16B 50/30; G06F 17/18

(57) ABSTRACT

A method for analysis of transcriptional aberrations and molecular diagnostic of genetic diseases includes receiving ribonucleic acid, RNA, related data; calculating a probability λₜ of an error-free splicing for a coding transcript t based on the RNA data; calculating the count-per-million (CPM) normalized xₜ for the coding transcript t based on the RNA data; calculating an omega index based on a product of the probability λₜ and the CPM normalized xₜ for a gene g of the human genome; and determining that the gene g is a candidate for a genetic disorder.

### CROSS-REFERENCE TO RELATED APPLICATION

This application claims priority to U.S. Provisional Patent Application No. 62/813,301, filed on Mar. 4, 2019, entitled “NEW METHOD TO QUANTIFY AND EVALUATE THE ACTIVITY OF GENES USING RNA-SEQ DATA,” the disclosure of which is incorporated herein by reference in its entirety.

### BACKGROUND

### Technical Field

Embodiments of the subject matter disclosed herein generally relate to a method for RNA-seq quantification for estimating an abundance level of functional mRNAs for determining genes with deleterious transcriptional aberrations.

### Discussion of the Background

RNA-seq has been adopted to study a wide range of biological problems. Recent developments have shown the promising use of RNA sequencing (RNA-seq) data in clinical diagnostics. Importantly, as transcriptomics (i.e., a set of all RNA molecules in one cell or a population of cells) helps improve the interpretation of functional consequences of underlying genetic variants, the use of RNA-seq data can lead to the detection of causative genes of genetic disorders that genome-based diagnostics alone has failed to identify.

A step in the application of RNA-seq data is the use of appropriate quantification methods suitable for specific downstream analysis [1], [2]. In transcriptome-based diagnostics, RNA-seq data are typically quantified to detect splicing outliers through analysis of differential splicing events between cases and controls [3], [4]. Splicing is a form of RNA processing in which a newly made precursor messenger RNA (pre-mRNA) transcript is transformed into a mature messenger RNA (mRNA). During splicing, introns (i.e., non-coding regions) are removed and exons (e.g., coding regions) are joined together. For those eukaryotic genes that contain introns, splicing is usually required in order to create an mRNA molecule that can be translated into a protein. A splicing outlier corresponds to a gene that is aberrantly expressed with abnormal splicing patterns and thus, such an mRNA molecule is an undesirable one.

However, a large number of unannotated splicing junctions, often found in the RNA-seq data, makes the discovery of the causative genes solely from the quantification of splicing junctions challenging. A splicing junction can be annotated or unannotated. An annotated splicing junction is a junction found in a normal mRNA molecule for which there is prior information, and thus, such a junction is known and expected to happen in a certain fashion. However, an unannotated splicing junction is not known, and there is no prior information about it.

Furthermore, the degradation of aberrant transcripts via the activation of the nonsense-mediated decay (NMD) pathway can complicate the prediction of causative genes from the splicing outliers. The NMD is an mRNA quality-control mechanism that typifies all eukaryotes examined to date. The NMD surveys the newly synthesized mRNAs and degrades those that harbor a premature termination codon (PTC), thereby preventing the production of truncated proteins that could result in disease in humans. To take into account the degradation of such deleterious variants, gene expression outliers can be identified via differential expression analysis between cases and controls. A gene expression outlier is undesirable in the gene expression process.

Indeed, the integration of these two separate types of transcriptional aberration analysis has shown to increase the diagnostic rate [5], [6]. However, because the existing RNA-seq quantifications used for aberrant expression analysis in these previous studies focus on the overall abundance levels of all transcripts [5] to [7], they substantially overestimate the abundance level of the functional mRNAs (i.e., mRNAs that produce proteins) when nonfunctional, aberrant, transcripts with splicing error are present. For this reason, the traditional methods underestimate the signal for detecting true causative genes.

Thus, there is a need for a new method or measure for determining a more accurate abundance level of the transcriptional aberrations.

### BRIEF SUMMARY OF THE INVENTION

According to an embodiment, there is a method for analysis of transcriptional aberrations and molecular diagnostic of genetic diseases. The method includes a step of receiving ribonucleic acid, RNA, related data, a step of calculating a probability λₜ of an error-free splicing for a coding transcript t based on the RNA data, a step of calculating the count-per-million (CPM) normalized xₜ for the coding transcript t based on the RNA data, a step of calculating an omega index based on a product of the probability λₜ and the CPM normalized xₜ for a gene g of the human genome, and a step of determining that the gene g is a candidate for a genetic disorder.

According to another embodiment, there is a computing device for analysis of transcriptional aberrations and molecular diagnostic of genetic diseases. The computing device includes an interface configured to receive ribonucleic acid, RNA, related data, and a processor connected to the interface and configured to calculate a probability λₜ of an error-free splicing for a coding transcript t based on the RNA data, calculate the count-per-million (CPM) normalized xₜ for the coding transcript t based on the RNA data, calculate an omega index based on a product of the probability λₜ and the CPM normalized xₜ for a gene g of the human genome, and determine that the gene g is a candidate for a genetic disorder.

### BRIEF DESCRIPTION OF THE DRAWINGS

For a more complete understanding of the present invention, reference is now made to the following descriptions taken in conjunction with the accompanying drawings, in which:

FIG. 1 illustrates a method for analysis of transcriptional aberrations and molecular diagnostic of genetic diseases;

FIG. 2 illustrates the computation of error-free splicing rate for a donor-acceptor pair in a coding transcript;

FIG. 3 illustrates the computation of the abundance level of annotated mRNA transcript for a given coding gene;

FIG. 4 illustrates the omega measure, which partitions the abundance level of each coding gene into those for the annotated, splicing error-free mRNAs and unannotated, cryptic mRNAs;

FIGS. 5A to 5H illustrate abundance-based, tissue-type, clustering using the omega measure and a traditional method;

FIGS. 6A to 6E are histograms showing the number of expression outliers based on various cut-off threshold values and calculated with the omega measure and the traditional method;

FIG. 7 illustrates the ranking of the causative genes of five cases based on a low expression outlier analysis with the omega measure and the traditional method;

FIG. 8 is a flowchart of a method for analysis of transcriptional aberrations and molecular diagnostic of genetic diseases; and

FIG. 9 is a schematic diagram of a computing device in which one or more of the methods discussed herein can be implemented.

### DETAILED DESCRIPTION OF THE INVENTION

The following description of the embodiments refers to the accompanying drawings. The same reference numbers in different drawings identify the same or similar elements. The following detailed description does not limit the invention. Instead, the scope of the invention is defined by the appended claims.

Reference throughout the specification to “one embodiment” or “an embodiment” means that a particular feature, structure or characteristic described in connection with an embodiment is included in at least one embodiment of the subject matter disclosed. Thus, the appearance of the phrases “in one embodiment” or “in an embodiment” in various places throughout the specification is not necessarily referring to the same embodiment. Further, the particular features, structures or characteristics may be combined in any suitable manner in one or more embodiments.

According to an embodiment, a novel measure, called herein the omega measure or the omega index, is introduced and this measure evaluates transcriptional aberrations by focusing on the portions of the functional mRNAs rather than the overall mRNA abundance levels. This means that the omega measure is an RNA-seq quantification with the ability to more accurately estimate the abundance level of functional mRNAs, which is believed to be more suited for the analysis of true gene expression and splicing outliers. The omega measure (observed mRNA's error-free gene-level abundance) is a between-sample RNA-seq quantification that estimates the gene-level abundances of splicing-error-free mRNA transcripts annotated to code for functional proteins. The term “code” herein is used to distinguish over the mRNAs that are not resulting into a functional protein. By using clinically established cases of genetic disorders, the inventors have demonstrated, as discussed later, that the novel measure omega is a practical application in the genome field capable to intensify the signal for deleterious transcriptional aberrations, to narrow down the search space for the prediction of causative genes.

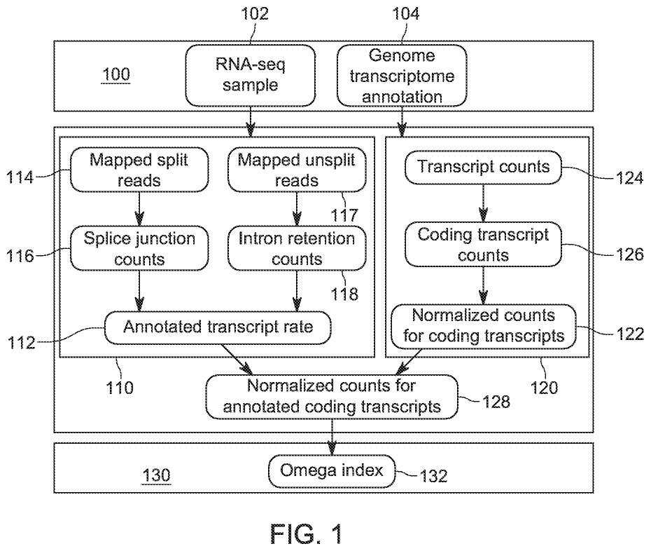

The omega measure estimates the relative abundance of the mRNA transcripts produced through error-free splicing processes, i.e., functional mRNA transcripts. FIG. 1 illustrates a workflow for the omega index or measure. To compute the omega measure according to this workflow, an RNA-seq sample 102 and a genome transcriptome annotation 104 are received as input in step 100. The later data can be obtained from any known database. The method estimates in steps 110 and 120 the error-free splicing rate and the abundance level of each coding transcript specified in a given transcriptome annotation. An error-free splicing event is defined herein to be that event without any cryptic splicing events based on the annotation in use. A cryptic splicing event is not an annotated event and may also reflect a DNA error that propagates to the RNA.

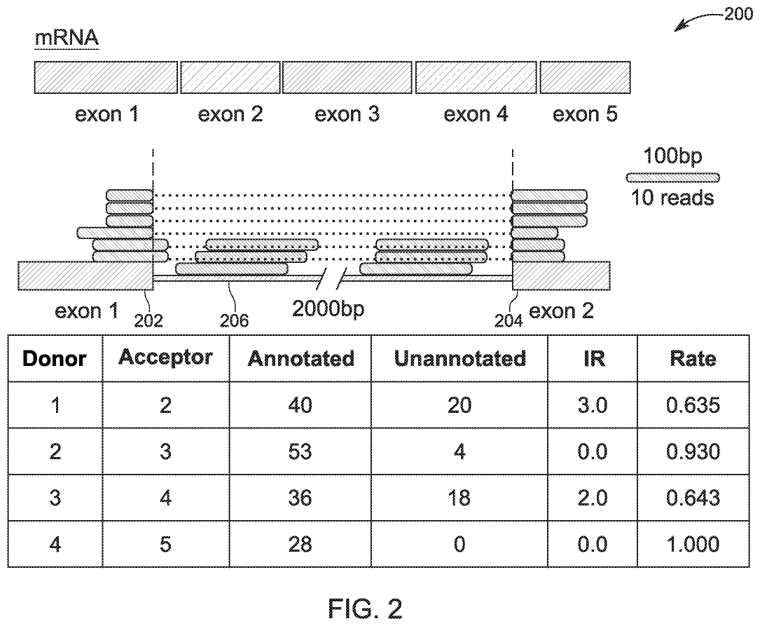

The method computes the probability of error-free splicing events for each pair of consecutive exons in each coding transcript based on observed splicing and intron retention events as illustrated in FIG. 2. For each donor 202—acceptor 204 pair, the method estimates the fraction of error-free mRNA transcripts. Note that the introns 206 (nucleotide sequence in the gene) that are present between the plural exons (another nucleotide sequence in the gene) in the genome are removed by the RNA to create the mature RNA, i.e., mRNA 200 in FIG. 2. The mRNA 200 in FIG. 2 is the transcript T1 of a gene G, and the mRNA transcript includes only exons 202, 204, and no introns 206.

FIG. 2 also shows an example of the calculated error-free splicing rate for each donor-acceptor pair in a coding transcript for annotated and unannotated splicing, and the associated intron retention (IR).

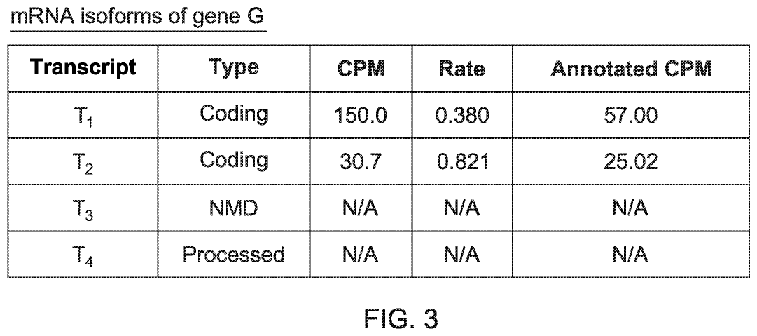

By using the splice junction counts and the intron retention counts values as weights in the count-per-million (CPM) measure of the mRNA transcripts for each coding gene G, the method is able to compute in step 130 the omega measure 132. FIG. 3 illustrates plural transcripts T1 to T4, some of which are coding and some not, their corresponding CPM measure, rate, and the annotated CPM. Note that the values for the non-coding transcripts are not calculated for the CPM, rate and annotated CPM as these transcripts are not used for determining the measure omega. The decision to use the CPM as the normalization came from the fact that the omega measure is configured to compare the abundance of a given gene between samples (i.e., case and control) and not to compare abundances of different genes within a sample.

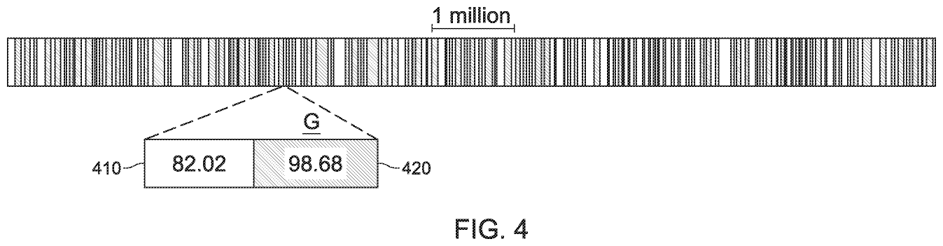

The omega measure allows one to partition the expression level of each coding gene G into an annotated value 410, corresponding to the omega measure, as illustrated in FIG. 4, which represents the splicing error-free, functional forms, and an unannotated value 420, which represents aberrant, nonfunctional forms and has a value given by the CPM minus the omega index. While the definition of the error-free splicing depends on a particular annotation that is in use and may not capture every error-free RNA splicing events that constitute the functional transcripts, the omega measure can better estimate the relative abundance level of functional mRNAs between samples using the same genome and transcriptome annotation consistently across the board.

Assuming that the functional mRNAs' abundances play an important role in controlling the phenotypic state of the cell, the omega measure is expected to differentiate RNA-seq samples based on their phenotypic characteristics. Indeed, clustering analysis of the GTEx data (see the Consortium G, (2013), The Genotype-Tissue Expression (GTEx) project. Nature genetics 45: 580-585) showed that the omega measure outperformed the transcript-per-million (TPM) quantification in differentiating RNA-seq samples with different tissue types, as discussed later.

The method for calculating the omega measure 132 illustrated in FIG. 1 is now discussed in more detail. With regard to this figure, after step 100 of receiving the input data 102 and 104 noted above, the method aligns in the same step 100 the reads to hg38 (using GENCODE 25, which is available at https://www.gencodegenes.org/human/release_25.html) using STAR 2.6 with the two-pass option (see, for example, Dobin A, Davis C A, Schlesinger F, Drenkow J, Zaleski C, et al., (2013), STAR: ultrafast universal RNA-seq aligner. Bioinformatics (Oxford, England) 29: 15-21). It is noted that various steps in this method rely on well-known algorithms that one skilled in this art is familiar with, and for this reasons, those algorithms are just listed herein, but they are not discussed in detail, as they are not an essential part of the invention. In fact, any known algorithm disclosed herein can be replaced with similar algorithms known in the art. For those that are not familiar with these codes, a reference is provided after each such algorithm, but such references are not considered to be essential for the invention. This embodiment considers only reads mapped to chromosomes 1-22 and X in sub-step 114.

To count the frequency of each splicing junction in sub-step 116, the method computed the coverage of the observed introns from the uniquely mapped split reads of sub-step 114, using SAMtools (see, for example, Li H, Handsaker B, Wysoker A, Fennell T, Ruan J, et al., (2009), The sequence alignment/map format and SAMtools. Bioinformatics (Oxford, England) 25: 2078-2079) and BEDTools (see, for example, Quinlan A R, Hall I M, (2010), BEDTools: a flexible suite of utilities for comparing genomic features. Bioinformatics (Oxford, England) 26: 841-842).

To classify each observed junction as annotated or unannotated in sub-step 112, the method computes in this embodiment a distance of each observed splicing junction from the closest donor and acceptor sites using the location of the annotated exons in GENCODE 25. To analyze in sub-step 118 the intron retention events, the method uses SAMtools and BEDTools to count the number of unsplit reads, which are determined in sub-step 117 based on the input 104 in step 100, within annotated intronic regions and multiples the counts by the ratio of the read length to the corresponding intron size.

Given these measures, a Markovian assumption is made and the probability λₜ of splicing-error free, transcription rate for each coding transcript t is computed in sub-step 112, by using the formula:

$${λ}_{t} = {\prod }_{i ∈ {J}_{t}} \frac{{a}_{i} + ɛ}{{a}_{i} + {u}_{i} + i {r}_{i} + ɛ} , \qquad ( 1 )$$

where Jₜ is a set of splice junctions for transcript t, aᵢ is the count of the annotated splice junction i (calculated in sub-step 116), uᵢ is the count of the unannotated splice junction i (calculated in sub-step 116), irᵢ is the normalized count for the intron retention (calculated in sub-step 118) within the annotated splicing region, and ε is a small constant.

The step 120 discussed above, which achieves the transcript count normalization, includes a sub-step 124 of determining the transcript counts and a sub-step 126 of mapping the RNA-seq reads to a reference transcriptome for hg38 (GEN-CODE 25) using kallisto (Bray N L, Pimentel H, Melsted P, Pachter L, (2016), Near-optimal probabilistic RNA-seq quantification. Nature biotechnology 34: 525-527) to estimate the transcript counts. To compute the CPM, i.e., the normalized counts for coding transcripts, the method selects those transcripts that are annotated as protein-coding from chromosomes 1-22 and X and normalizes their counts so that the sum is fixed to 1 million.

To compute the TPM, for comparison purposes, the counts of the selected transcripts were divided by their lengths and then normalized the length-normalized counts to have the sum to be 1 million. To obtain the gene-level CPM and TPM for each coding gene, the normalized values of its coding transcripts were summed up. The normalized count for the annotated coding transcripts was calculated in sub-step 128, based on the results from sub-steps 112 and 122.

Based on the annotated transcript rate from sub-step 112 and the CPM from sub-step 122, the omega index is defined as:

$${ω}_{g} = {\sum }_{t ∈ {T}_{g}} {λ}_{c} {x}_{t} , \qquad ( 2 )$$

where T_{g} is a set of coding transcripts annotated for gene g, λₜ is the annotated, error-free splicing for transcript t, calculated in sub-step 112, and xₜ is the CPM for the coding transcript t calculated in sub-step 122.

Based on the method illustrated in FIG. 1 and discussed above, the gene-level abundance of normal mRNAs for a gene from the genome can be calculated more accurately than the existing methods.

To compare the prediction power of the omega measure with the existing measures, the following definition has been used to determine the low expression outliers. The gene-level mRNA abundance levels of the cases and the GTEx dataset were quantified using the traditional TPM measure and the novel omega measure, and they were then log-transformed with base 10. For each gene g in the coding gene set G, let Q1_{g} and Q2_{g} be the 1st quartile and the 2nd quartile of the log-transformed gene-level mRNA abundance distribution of the GTEx cohort for a given cell type, respectively, and y_{g} be the log-transformed mRNA abundance level of the gene g from a case. Then, the low expression outliers are computed using a_{g} defined as follows:

$${a}_{g} = \frac{Q {1}_{g} - {y}_{g}}{Q {2}_{g} - Q {1}_{g}} . \qquad ( 3 )$$

Using a threshold parameter θ, the low expression outliers are defined to be a subset of G in which each gene g satisfies a_{g}≥θ. Genes with no detectable promoter activity, i.e., gene-level TPM=0, are excluded from the pool of potential expression outliers.

Based on this measure, the prediction of the omega index has been tested as now discussed. RNA samples of either fibroblasts or lymphocytes from five patients with clinically established autosomal recessive diseases were prepared. The samples were sequenced for obtaining the RNA-seq. The quality of each RNA sample was determined based on its RNA Integrity Number (RIN) and ensured that RNA-seq data were generated from a high-quality RNA sample (RIN 8.0) for each case. Then the sequencing libraries were prepared and paired-end 150 bp reads were generated. The GTEx RNASeq samples were downloaded from publicly available data (e.g., Consortium G, (2013), The Genotype-Tissue Expression (GTEx) project. Nature genetics 45: 580-585) for the blood and skin tissue types from the Database of Genotypes and Phenotypes with RIN≥6 (n=1,572) and transformed into the fastq format using SRA Toolkit (see, e.g., https://www.ncbi.nlm.nih.gov/sra/docs/toolkitsoft). These RNA-seq datasets consist of paired-end 75 bp reads.

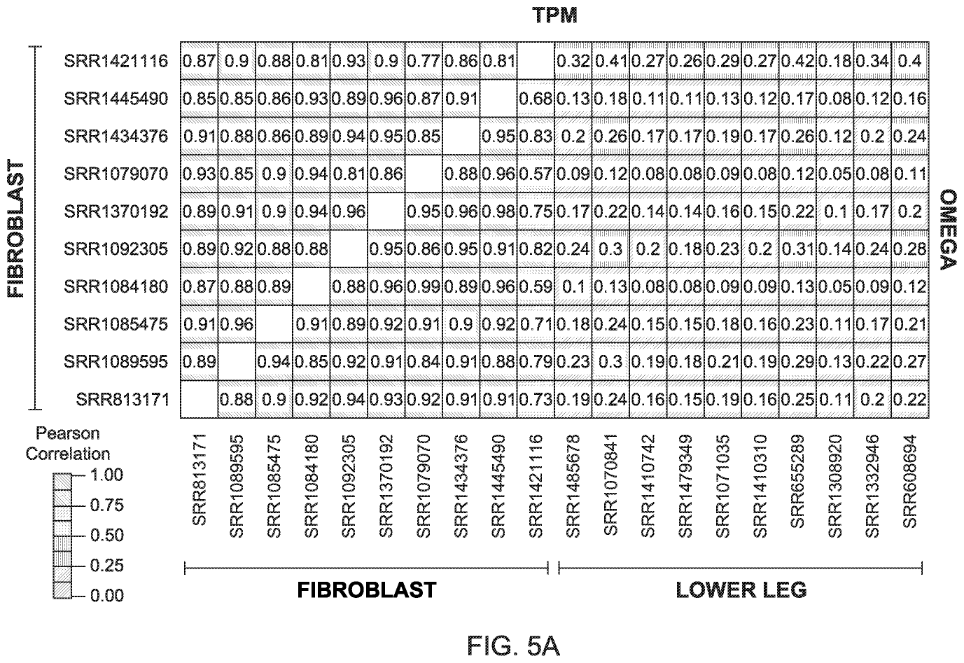

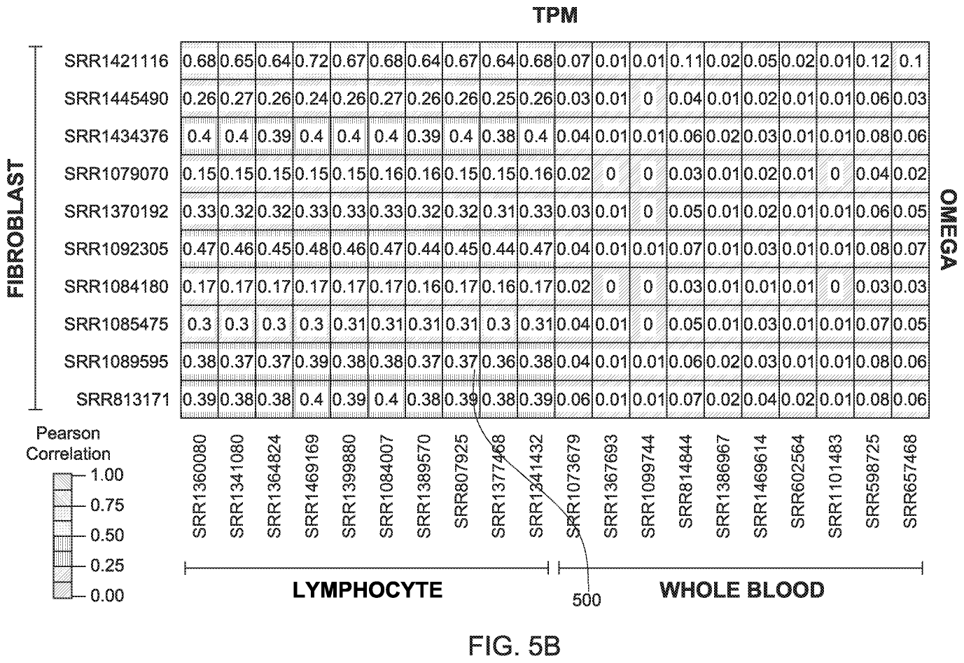

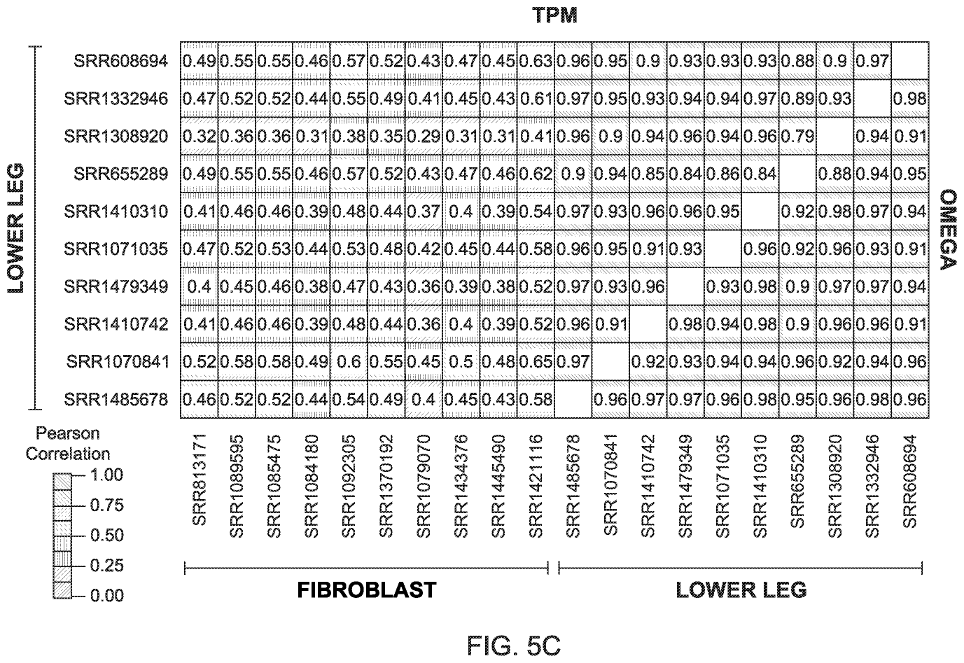

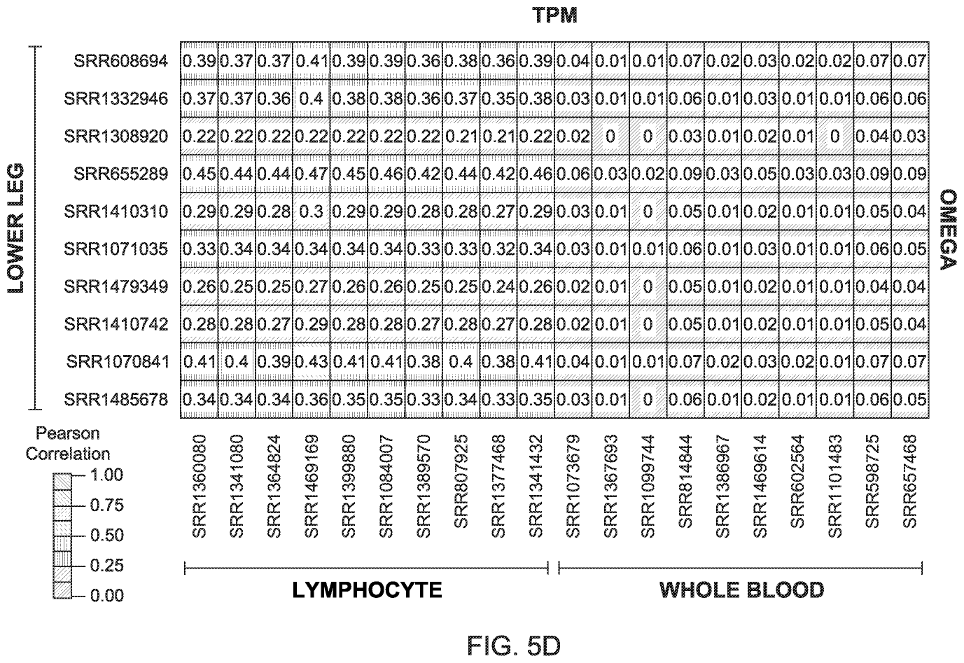

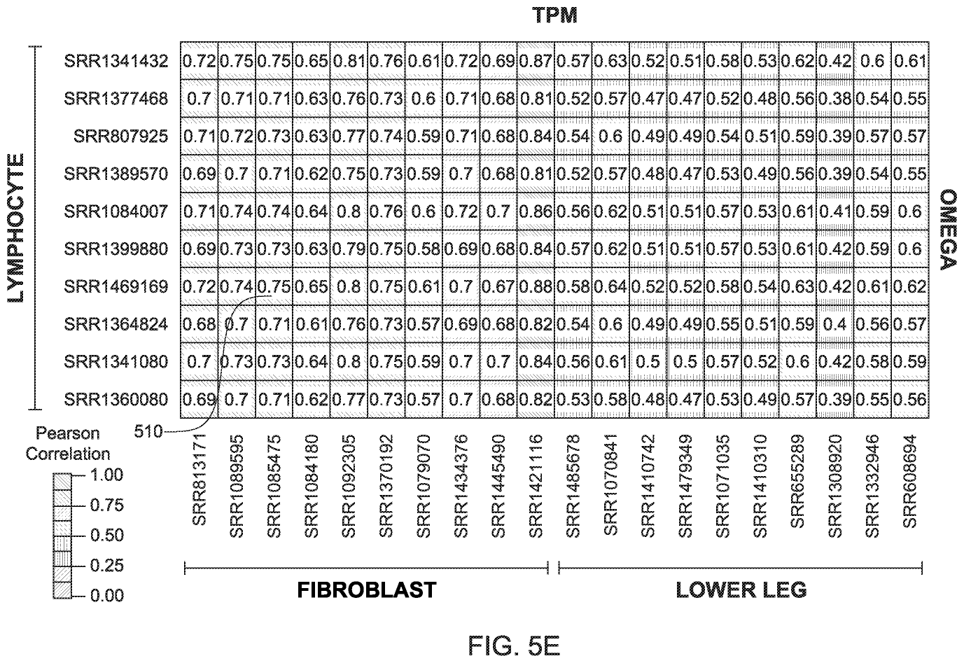

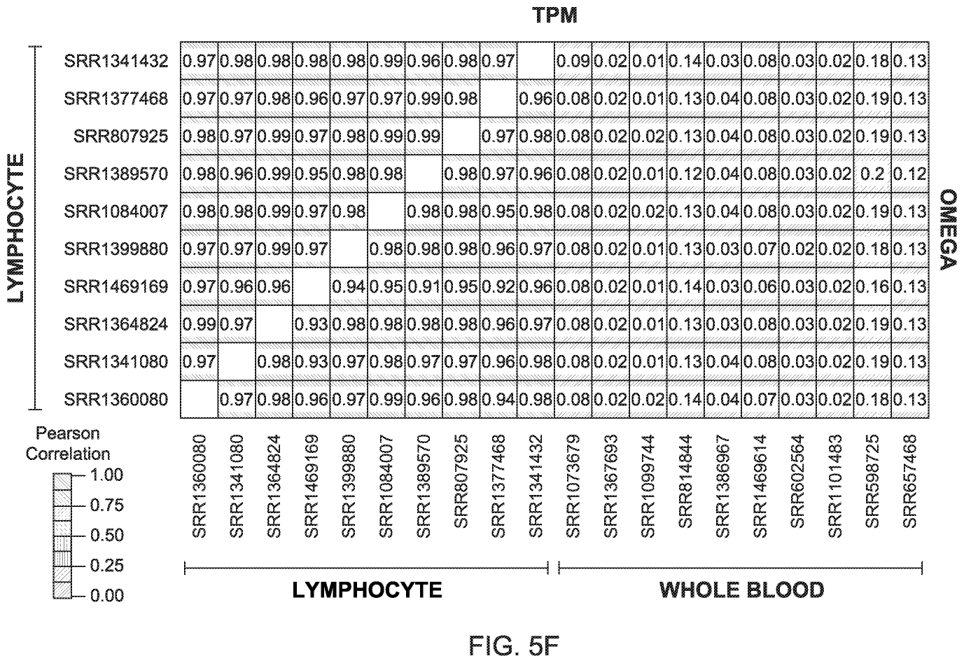

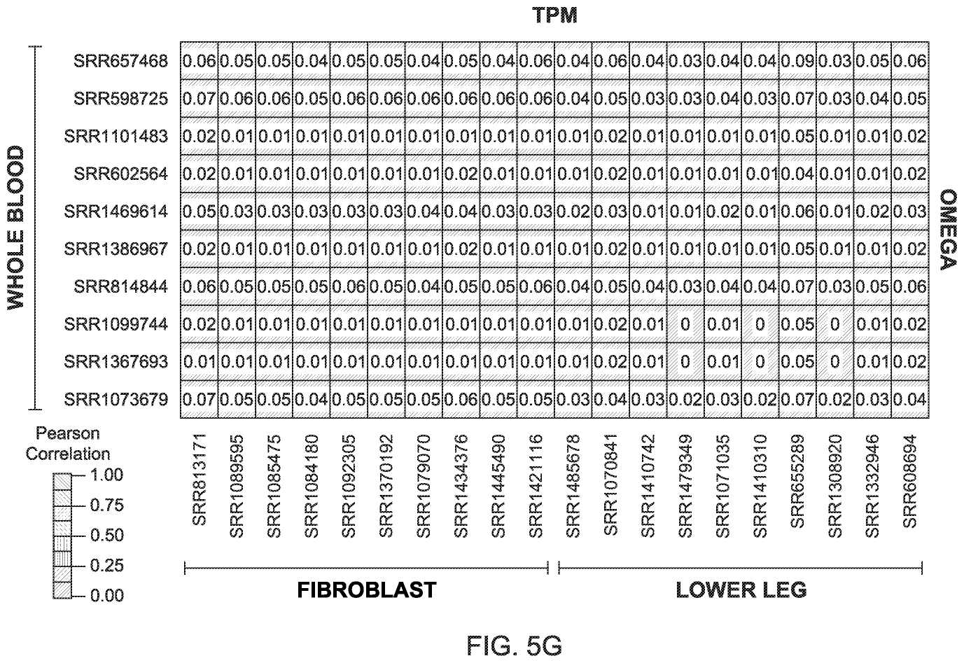

To investigate the value of the novel RNA-seq quantification in clinical diagnostics, the omega measure and TPM were applied to the five clinically established cases of distinct autosomal recessive disorders with known causal transcriptional aberrations as a case study. For this comparison, the GTEx RNA-seq data was used as control data (skin fibroblast: n=305 and lymphocyte: n=b 134) and the low expression outliers were determined based on equation (3), out of 19,688 coding genes in the five diagnosed cases. Here, the desirable outcome from the RNA-seq quantification is the selection of a small set of expression outliers that contain the true disease gene. Because each of these cases has a large number of unique splicing junction sites that are not found in the corresponding cell type of the GTEx cohort, as illustrated in FIGS. 5A to 5H, the detection of splicing outliers alone cannot lead to meaningful sets of disease gene candidates, thus requiring to filter the given candidates based on different criteria, such as those based on mRNA abundance levels.

Note that in FIGS. 5A to 5H the upper-left triangular matrix shows the pair-wise correlation obtained with the TPM method, while the lower-right triangular matrix shows the pair-wise correlation obtained with the omega measure. Ten RNA-seq samples with RIN≥8 were randomly selected from each of 4 tissue types in blood and skin in the GTEx dataset, and pair-wise correlation of these 40 samples was computed using the omega and TPM methods. While both the omega and TPM based methods were able to have high correlation values for samples of the same tissue type, the omega measure was able to discriminate samples of different tissue types far better, as noted when comparing areas 500 and 510 in FIGS. 5B and 5E. Specifically, while the TPM method could not clearly differentiate samples of lymphocytes from those of fibroblasts and lower leg, the omega measure was able to classify those samples based on their tissue types.

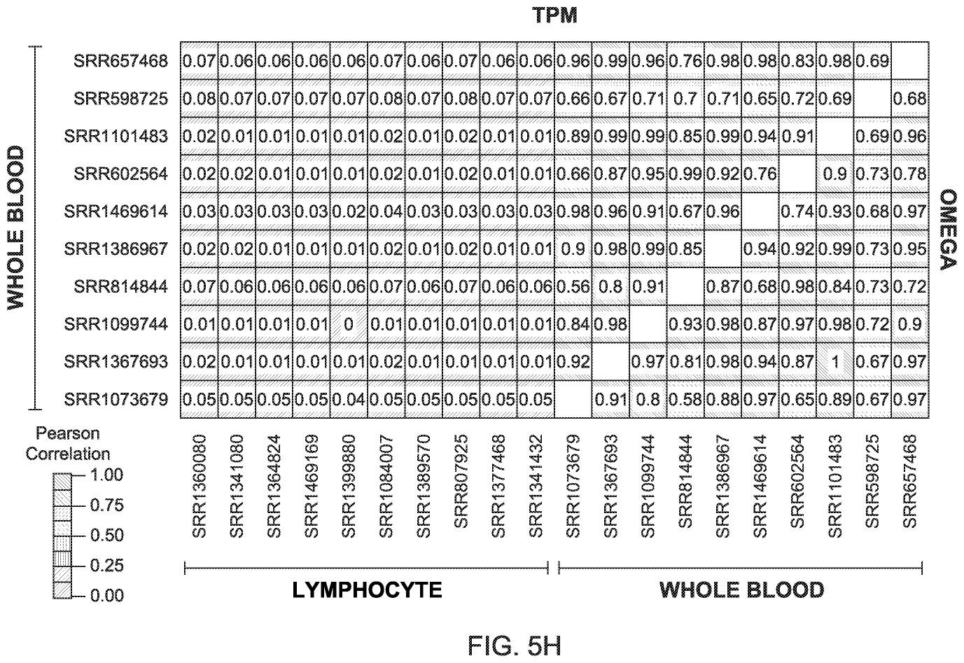

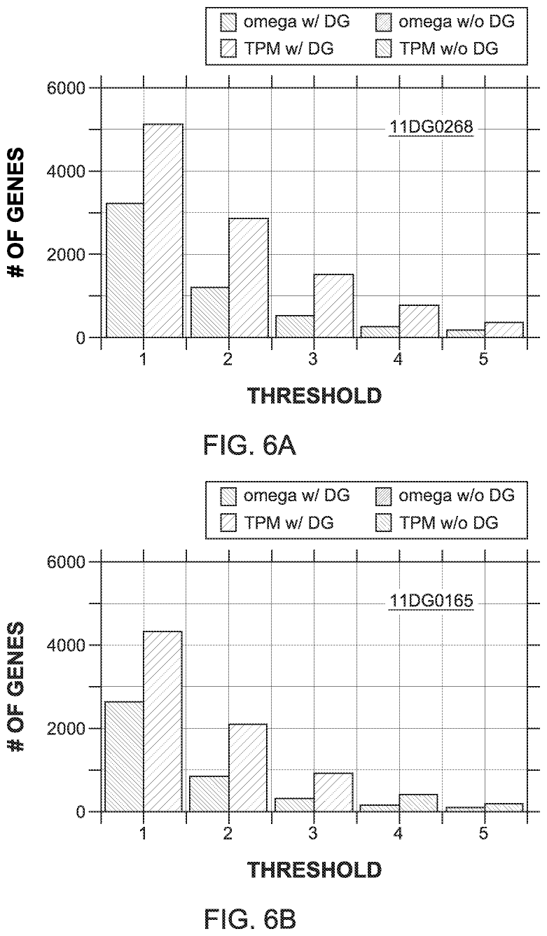

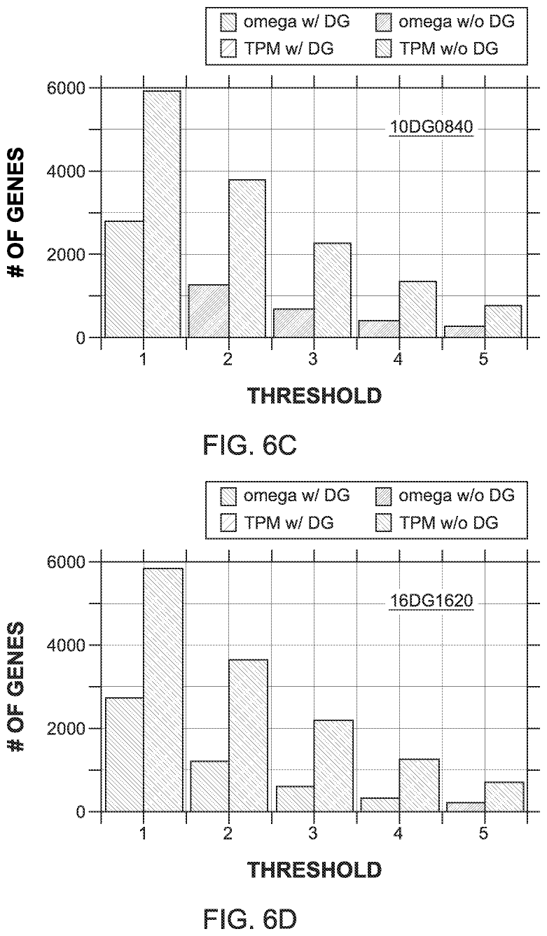

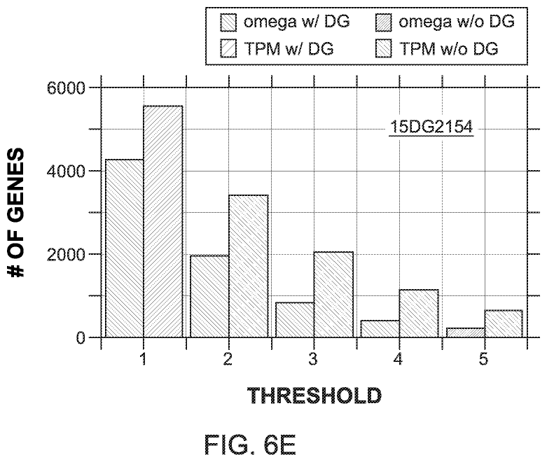

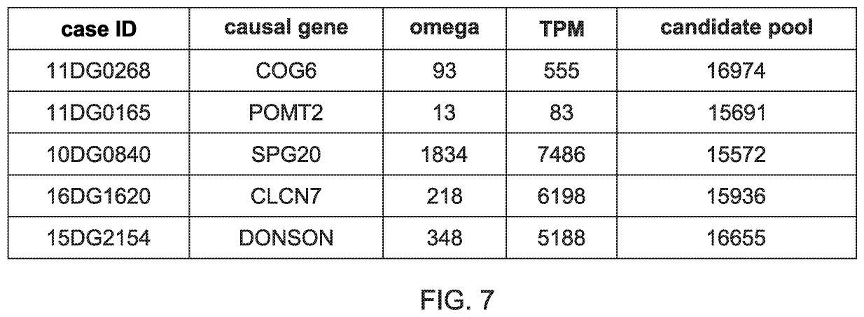

The inventors have found that, in all five cases, the omega index was able to generate smaller sets of outliers that contain the true causal genes than the TPM method, as shown in FIGS. 6A to 6E. FIGS. 6A to 6E show histograms indicating the number of expression outliers calculated with the omega measure and the TPM method, based on various cut-off threshold values, for the five diagnosed cases. The term DG means disease gene, and each bar is coded based on the presence or absence of the disease-causing gene in the expression outliers. For example, for genes 11 DG0268 and 11DG0165, while both omega and TPM identified the true causal genes in small sets of outliers, the omega measure was able to further filter out the non-causal genes. In addition to the ability to reduce the number of false-positive genes, the omega measure also improved the candidate ranking of true casual genes of these genetic diseases, as illustrated in the table shown in FIG. 7. Specifically, CLCN7, the causative gene of 16DG1620, was ranked 218th by the omega measure and 6,198th by the TPM method out of 15,936 candidates based on the low expression score defined in equation (3), while DONSON, the causative gene of 15DG2154, was ranked 348th by the omega measure and 5,188th by the TPM measure out of 16,655 genes. This indicates that the signal to detect the true casual genes can be substantially intensified by the novel omega measure, which recognizes abnormal transcripts that escaped the NMD and were left in the RNA samples.

In this regard, the comparison of the mRNA abundances of the causative genes between the omega measure and the TPM method highlights that the consideration of the error-free transcripts allowed the omega measure to strengthen the discriminability of the disease-causing gene between the cases and the controls. While the above discussion illustrated the application of the omega measure to the autosomal recessive diseases, this quantification is also expected to be suitable for molecular diagnostics of other types of genetic diseases, as well as complex diseases such as cancer.

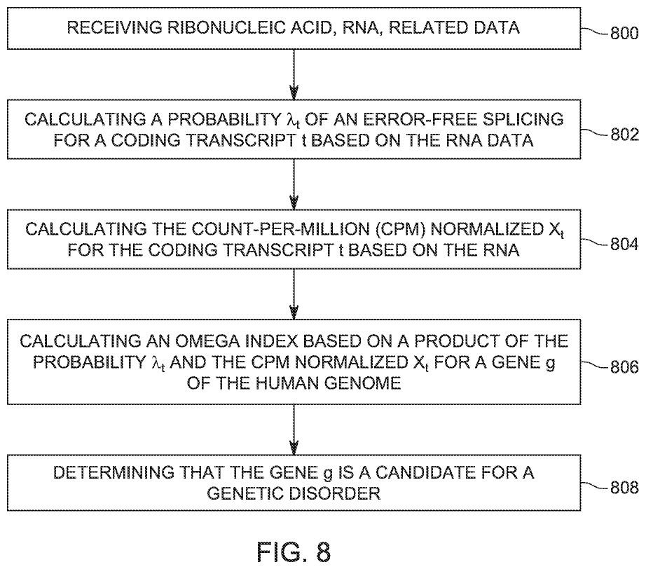

A method for analysis of transcriptional aberrations and molecular diagnostic of genetic diseases is now discussed with regard to FIG. 8. The method includes a step 800 of receiving ribonucleic acid, RNA, related data, a step 802 of calculating a probability λₜ of an error-free splicing for a coding transcript t based on the RNA data, a step 804 of calculating the count-per-million (CPM) normalized xₜ for the coding transcript t based on the RNA, a step 806 of calculating an omega index based on a product of the probability λₜ and the CPM normalized xₜ for a gene g of the human genome, and a step 808 of determining that the gene g is a candidate for a genetic disorder. The omega index quantifies an abundance level of functional mRNA and the RNA data includes samples of RNA-seq data and genome transcriptome annotation data.

In one application, the step of calculating a probability λₜ of an error-free splicing for a coding transcript t includes calculating a ratio of (1) a count of an annotated splice junction and (2) a sum of (i) the count of the annotated splice junction, (ii) a count of unannotated splice junction, and (iii) a normalized count of an intron reduction within the annotated splicing region. The ratio for the junction i is multiplied with corresponding ratios of other junctions that belong to a set of splicing junctions for the transcript t to calculate the probability λₜ.

The step of calculating the count-per-million (CPM) normalized xₜ for the coding transcript t may include determining the transcript counts; selecting that transcripts that are annotated as protein-coding to obtain coding transcript counts; and normalizing the coding transcript counts so that a sum of the coding transcript counts is one million. The step of calculating the omega index includes calculating a product of the probability λₜ and the CPM normalized xₜ for each transcript t, which is part of a set T_{g} of coding transcripts annotated for the gene g. In one application, a transcript is determined to be annotated by calculating a distance of each observed splicing junction from a closest donor and acceptor sites using a location of an annotated exon of the RNA.

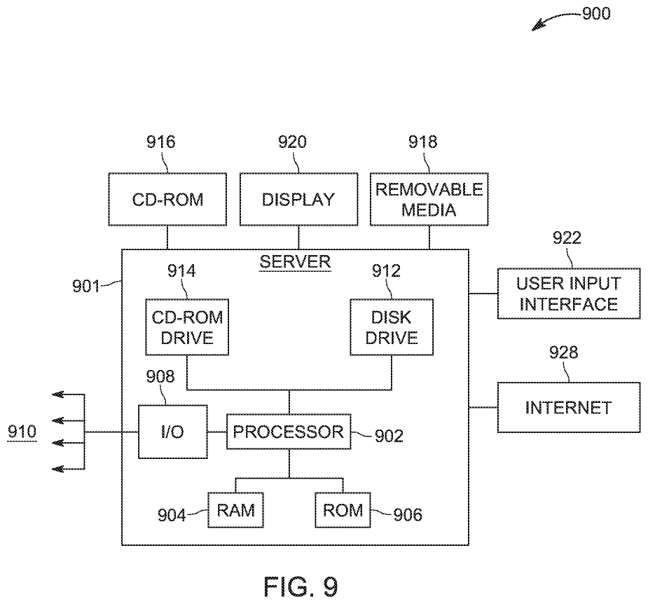

The above-discussed procedures and methods may be implemented in a computing device as illustrated in FIG. 9. Hardware, firmware, software or a combination thereof may be used to perform the various steps and operations described herein. Computing device 900 of FIG. 9 is an exemplary computing structure that may be used in connection with such a system.

Exemplary computing device 900 suitable for performing the activities described in the embodiments may include a server 901. Such a server 901 may include a central processor (CPU) 902 coupled to a random access memory (RAM) 904 and to a read-only memory (ROM) 906. ROM 906 may also be other types of storage media to store programs, such as programmable ROM (PROM), erasable PROM (EPROM), etc. Processor 902 may communicate with other internal and external components through input/output (I/O) circuitry 908 and bussing 910 to provide control signals and the like. Processor 902 carries out a variety of functions as are known in the art, as dictated by software and/or firmware instructions.

Server 901 may also include one or more data storage devices, including hard drives 912, CD-ROM drives 914 and other hardware capable of reading and/or storing information, such as DVD, etc. In one embodiment, software for carrying out the above-discussed steps may be stored and distributed on a CD-ROM or DVD 916, a USB storage device 918 or other form of media capable of portably storing information. These storage media may be inserted into, and read by, devices such as CD-ROM drive 914, disk drive 912, etc. Server 901 may be coupled to a display 920, which may be any type of known display or presentation screen, such as LCD, plasma display, cathode ray tube (CRT), etc. A user input interface 922 is provided, including one or more user interface mechanisms such as a mouse, keyboard, microphone, touchpad, touch screen, voice-recognition system, etc.

Server 901 may be coupled to other devices, such as sources, detectors, DNA and RNA decoders, etc. The server may be part of a larger network configuration as in a global area network (GAN) such as the Internet 928, which allows ultimate connection to various landline and/or mobile computing devices.

The disclosed embodiments provide a method for estimating the abundance level of functional mRNAs in a between-sample RNA-seq quantification, for analysis of transcriptional aberrations and molecular diagnostic of genetic diseases. It should be understood that this description is not intended to limit the invention. On the contrary, the embodiments are intended to cover alternatives, modifications and equivalents, which are included in the spirit and scope of the invention as defined by the appended claims. Further, in the detailed description of the embodiments, numerous specific details are set forth in order to provide a comprehensive understanding of the claimed invention. However, one skilled in the art would understand that various embodiments may be practiced without such specific details.

Although the features and elements of the present embodiments are described in the embodiments in particular combinations, each feature or element can be used alone without the other features and elements of the embodiments or in various combinations with or without other features and elements disclosed herein.

This written description uses examples of the subject matter disclosed to enable any person skilled in the art to practice the same, including making and using any devices or systems and performing any incorporated methods. The patentable scope of the subject matter is defined by the claims, and may include other examples that occur to those skilled in the art. Such other examples are intended to be within the scope of the claims.

### REFERENCES

[1] Robinson M D, Oshlack A (2010) A scaling normalization method for differential expression analysis of RNA-seq data. Genome biology 11: R25.
[2] Evans C, Hardin J, Stoebel D M (2018) Selecting between-sample rna-seq normalization methods from the perspective of their assumptions. Briefings in bioinformatics 19: 776-792.
[3] Vaquero-Garcia J, Barrera A, Gazzara M R, Gonz'alez-Vallinas J, Lahens N F, et al. (2016) A new view of transcriptome complexity and regulation through the lens of local splicing variations. eLife 5: e11752.
[4] Li Y I, Knowles D A, Humphrey J, Barbeira A N, Dickinson S P, et al. (2018) Annotation-free quantification of RNA splicing using LeafCutter. Nature genetics 50: 151-158.
[5] Kremer L S, Bader D M, Mertes C, Kopajtich R, Pichler G, et al. (2017) Genetic diagnosis of Mendelian disorders via RNA sequencing. Nature communications 8: 15824.
[6] Fr'esard L, Smail C, Ferraro N M, Teran N A, Li X, et al. (2019) Identification of rare-disease genes using blood transcriptome sequencing and large control cohorts. Nature medicine 25: 911-919.
[7] Cummings B B, Marshall J L, Tukiainen T, Lek M, Donkervoort S, et al. (2017) Improving genetic diagnosis in Mendelian disease with transcriptome sequencing. Science translational medicine 9.

What is claimed is:

1. A method for analysis of transcriptional aberrations and molecular diagnostic of genetic diseases, the method comprising:
receiving ribonucleic acid, RNA, related data;
calculating a probability λₜ of an error-free splicing for a coding transcript t based on the RNA data;
calculating the count-per-million (CPM) normalized xₜ for the coding transcript t based on the RNA data;
calculating an omega index based on a product of the probability λₜ and the CPM normalized xₜ for a gene g of the human genome; and
determining that the gene g is a candidate for a genetic disorder.

2. The method of claim 1, wherein the omega index quantifies an abundance level of functional mRNA.

3. The method of claim 1, wherein the RNA data includes samples of RNA-seq data and genome transcriptome annotation data.

4. The method of claim 1, wherein the step of calculating a probability λₜ of an error-free splicing for a coding transcript t comprises:
calculating a ratio of (1) a count of an annotated splice junction and (2) a sum of (i) the count of the annotated splice junction, (ii) a count of unannotated splice junction, and (iii) a normalized count of an intron reduction within the annotated splicing region.

5. The method of claim 4, wherein the ratio for the junction i is multiplied with corresponding ratios of other junctions that belong to a set of splicing junctions for the transcript t to calculate the probability λₜ.

6. The method of claim 4, wherein the step of calculating the count-per-million (CPM) normalized xₜ for the coding transcript t comprises:
determining the transcript counts;
selecting that transcripts that are annotated as protein-coding to obtain coding transcript counts; and
normalizing the coding transcript counts so that a sum of the coding transcript counts is one million.

7. The method of claim 6, wherein the step of calculating the omega index comprises:
calculating a product of the probability λₜ and the CPM normalized xₜ for each transcript t, which is part of a set T_{g} of coding transcripts annotated for the gene g.

8. The method of claim 6, wherein a transcript is determined to be annotated by calculating a distance of each observed splicing junction from closest donor and acceptor sites using a location of an annotated exon of the RNA.

9. The method of claim 1, wherein the omega measure partitions an abundance level of each coding gene into annotated, splicing error-free mRNAs and unannotated, cryptic mRNAs.

10. The method of claim 9, wherein the unannotated, cryptic mRNAs is indicative of an error in a corresponding gene.

11. A computing device for analysis of transcriptional aberrations and molecular diagnostic of genetic diseases, the computing device comprising:
an interface configured to receive ribonucleic acid, RNA, related data; and
a processor connected to the interface and configured to, calculate a probability λₜ of an error-free splicing for a coding transcript t based on the RNA data;
calculate the count-per-million (CPM) normalized xₜ for the coding transcript t based on the RNA data;
calculate an omega index based on a product of the probability λₜ and the CPM normalized xₜ for a gene g of the human genome; and
determine that the gene g is a candidate for a genetic disorder.

12. The computing device of claim 11, wherein the omega index quantifies an abundance level of functional mRNA.

13. The computing device of claim 11, wherein RNA data includes samples of RNA-seq data and genome transcriptome annotation data.

14. The computing device of claim 11, wherein the processor is further configured to:
calculate a ratio of (1) a count of an annotated splice junction and (2) a sum of (i) the count of the annotated splice junction, (ii) a count of unannotated splice junction, and (iii) a normalized count of an intron reduction within the annotated splicing region, as part of the probability λₜ of the error-free splicing for the coding transcript t.

15. The computing device of claim 14, wherein the ratio for the junction i is multiplied with corresponding ratios of other junctions that belong to a set of splicing junctions for the transcript t to calculate the probability λₜ.

16. The computing device of claim 14, wherein the processor is further configured to calculate, as part of calculating the count-per-million (CPM) normalized xₜ for the coding transcript t:
determining the transcript counts;
selecting that transcripts that are annotated as protein-coding to obtain coding transcript counts; and
normalizing the coding transcript counts so that a sum of the coding transcript counts is one million.

17. The computing device of claim 14, wherein the processor is further configured to calculate, as part of the step of calculating the omega index:
a product of the probability λₜ and the CPM normalized xₜ for each transcript t, which is part of a set T_{g} of coding transcripts annotated for the gene g.

18. The computing device of claim 14, wherein a transcript is determined to be annotated by calculating a distance of each observed splicing junction from closest donor and acceptor sites using a location of an annotated exon of the RNA.

19. The computing device of claim 11, wherein the omega measure partitions an abundance level of each coding gene into annotated, splicing error-free mRNAs and unannotated, cryptic mRNAs.

20. The computing device of claim 19, wherein the unannotated, cryptic mRNAs is indicative of an error in a corresponding gene.
